(19) United States
(12) Patent Application Publication
(10) Pub. No.: US 2024/0157174 A1
(43) Pub. Date: May 16, 2024

(54) HOUNSFIELD UNIT CALIBRATION IN RADIOTHERAPY SYSTEM

(71) Applicant: Siemens Healthineers International AG, Steinhausen (CH)

(72) Inventors: Mathias LEHMANN, Zürich (CH); Adam STRZELECKI, Dättwil (CH); Mathieu PLAMONDON, Glattbrugg (CH)

(73) Assignee: Siemens Healthineers International AG, Steinhausen (CH)

(21) Appl. No.: 18/509,251

(22) Filed: Nov. 14, 2023

Related U.S. Application Data
(60) Provisional application No. 63/425,943, filed on Nov. 16, 2022.

(51) Int. Cl. A61N 5/10; A61B 6/00

(52) U.S. Cl. CPC A61N 5/1071; A61B 6/583; A61N 5/1075; A61N 2005/1076; A61N 2005/1089

(57) ABSTRACT

A computer-implemented method of determining X-ray dose delivered to a region of patient anatomy includes: using a first imaging condition, generating a set of projection images of a region of patient anatomy; based on the set of projection images of the target volume, reconstructing a digital volume that includes a target volume disposed within the region of patient anatomy; based on the digital volume, determine current position of the target volume within the region of patient anatomy; delivering a treatment beam to the target volume while disposed at the current position; based on the first imaging condition, selecting a first calibration curve from a plurality of calibration curves, wherein each calibration curve in the plurality of calibration curves is associated with a different imaging condition; and based on the first calibration curve, determining an x-ray dose delivered to a portion of the target volume.

### CROSS-REFERENCE TO RELATED APPLICATION

The present application claims the benefit of U.S. Provisional Application No. 63/425,943, filed Nov. 16, 2022. The aforementioned U.S. Provisional Application, including any appendices or attachments thereof, is hereby incorporated by reference in its entirety.

### BACKGROUND

Unless otherwise indicated herein, the approaches described in this section are not prior art to the claims in this application and are not admitted to be prior art by inclusion in this section.

In radiation therapy, treatment planning based on computed tomography (CT) is the current gold standard. A CT scan for patient simulation represents the starting point for organ and tumor segmentation as well as for dose calculation. Current trends in image-guided radiotherapy are paving the way towards an increase in accuracy and precision of treatment delivery, shorter treatment times (e.g., hypo-fractionated and/or even flash therapy) and patient-individualized therapy concepts. Hence, an adaptive therapy workflow with plan adaptation based on the actual patient anatomy (“plan of the day”), which can change during the course of treatment, is a prerequisite to fulfill the demands of high-precision radiotherapy, whose goal is for improved tumor control and patient comfort with reduced toxicity. To be able to verify, track, control, and/or steer the dose delivery during treatment using a cone-beam computed tomography (CBCT) system, accurate mapping of CBCT values (e.g., Hounsfield units, or “HU”) to either relative electron density or mass density is required. However, such mapping generally varies for each specific radiation therapy system.

Conventional HU calibration and mapping procedures implemented in conventional CBCT reconstructors have some known systematic limitations in HU accuracy. Because matching of a CBCT image to a previously acquired treatment planning image does not require accurate absolute HU values, conventional HU calibration approaches have worked reasonably well for many radiotherapy systems with onboard CBCT imaging systems. However, for future simulation and treatment applications, quantitatively more accurate HU values are generally required to achieve a CBCT quality that is comparable to planning CTs from clinical CT scanners. In CBCT imaging, HU error may be small compared to more dominant artifacts, such as motion streaks, scatter correction errors, metallic artifacts, and the like. However, there are now intended use cases that can benefit from more accurate HU calibration procedures, such as simulation and treatment applications. In such use cases, accurate estimation of dosing based on HU values of a CBCT image require smaller HU error than is currently achievable.

### SUMMARY

According to various embodiments, the higher HU accuracy associated with clinical CT scanners can also be realized with a CBCT imaging system, such as an on-board CBCT imaging system that is in included in a radiotherapy system. In the embodiments, a plurality of system-specific calibration curves are generated for a particular CBCT imaging system, where each calibration curve is associated with a different imaging condition of the CBCT imaging system. In the embodiments, each imaging condition of the CBCT imaging system corresponds to a different combination of phantom (or patient anatomy) size, imaging energy, and/or imaging X-ray filtration. In some embodiments, when a digital volume is reconstructed based on projection images generated by the CBCT imaging system with a particular imaging condition, HU values of the digital volume are modified using the calibration curve associated with that particular imaging condition. The modified HU values are more accurately mapped to the physical characteristics, such as mass density or electron density. In such embodiments, the modified HU values are used to determine more accurate dosing. Alternatively or additionally, in some embodiments, a calibration curve associated with a particular imaging condition of the CBCT imaging system can be compared to a theoretical calibration curve as a consistency check that can indicate an imaging system error in the CBCT imaging system.

In some embodiments, a computer-implemented method of determining X-ray dose delivered to a region of patient anatomy includes: using a first imaging condition, generating a set of projection images of a region of patient anatomy; based on the set of projection images of the target volume, reconstructing a digital volume that includes a target volume disposed within the region of patient anatomy; based on the digital volume, determine current position of the target volume within the region of patient anatomy; delivering a treatment beam to the target volume while disposed at the current position; based on the first imaging condition, selecting a first calibration curve from a plurality of calibration curves, wherein each calibration curve in the plurality of calibration curves is associated with a different imaging condition; and based on the first calibration curve, determining an x-ray dose delivered to a portion of the target volume.

In some embodiments, a computer-implemented method of detecting an imaging system error in an X-ray imaging system includes: using a first imaging condition for the x-ray imaging system, generating a set of projection images of a phantom that includes one or more material inserts; based on the set of projection images of the phantom, reconstructing a digital volume that includes the one or more material inserts; for each of the one or more material inserts: determining a measured attenuation value based on the digital volume; and determining a corresponding theoretical attenuation value based on properties of the x-ray imaging system and on the first imaging condition for the x-ray imaging system; based on one or more of the measured attenuation values and one or more of the corresponding theoretical attenuation values, generating a first calibration curve for the X-ray imaging system associated with the first imaging condition; determining a difference between one or more characteristics of the first calibration curve and one or more characteristics of a second calibration curve that indicates a theoretically expected relationship between the one or more measured attenuation values and the one or more corresponding theoretical attenuation values when the one or more material inserts are imaged by the X-ray imaging system using the first imaging condition; and in response to the difference exceeding a threshold value, causing a warning indicator to be generated.

Further embodiments include a non-transitory computer-readable storage medium comprising instructions that cause a computer system to carry out one or more of the above methods, as well as a computer system configured to carry out one or more of the above methods.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of the present disclosure will become more fully apparent from the following description and appended claims, taken in conjunction with the accompanying drawings. These drawings depict only several embodiments in accordance with the disclosure and are, therefore, not to be considered limiting of its scope. The disclosure will be described with additional specificity and detail through use of the accompanying drawings.

FIG. 1A is a graph illustrating mass density as a function of measured HU value.

FIG. 1B is a graph illustrating electron density as a function of measured HU value.

FIG. 2 is a perspective view of a radiation therapy system that can beneficially implement various embodiments.

FIG. 3 schematically illustrates a drive stand and gantry of the radiation therapy system of FIG. 2, according to various embodiments.

FIG. 4 schematically illustrates a drive stand and a gantry of the radiation therapy system of FIG. 2, according to various embodiments.

FIG. 5 schematically illustrates a digital volume that is constructed based on projection images generated by one or more X-ray imagers included in the radiation therapy system of FIG. 2, according to various embodiments.

FIG. 6 sets forth a flowchart of a computer-implemented method for a radiotherapy system, according to one or more embodiments.

FIG. 7 schematically illustrates an example embodiment of an imaging phantom.

FIG. 8 illustrates a calibration curve, according to various embodiments.

FIG. 9 sets forth a flowchart of a computer-implemented method for a radiotherapy system, according to one or more embodiments.

FIG. 10 is an illustration of a computing device configured to perform various embodiments.

FIG. 11 is a block diagram of an illustrative embodiment of a computer program product for implementing one or more embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the disclosure, as generally described herein, and illustrated in the figures, can be arranged, substituted, combined, and designed in a wide variety of different configurations, all of which are explicitly contemplated and make part of this disclosure.

### Introduction

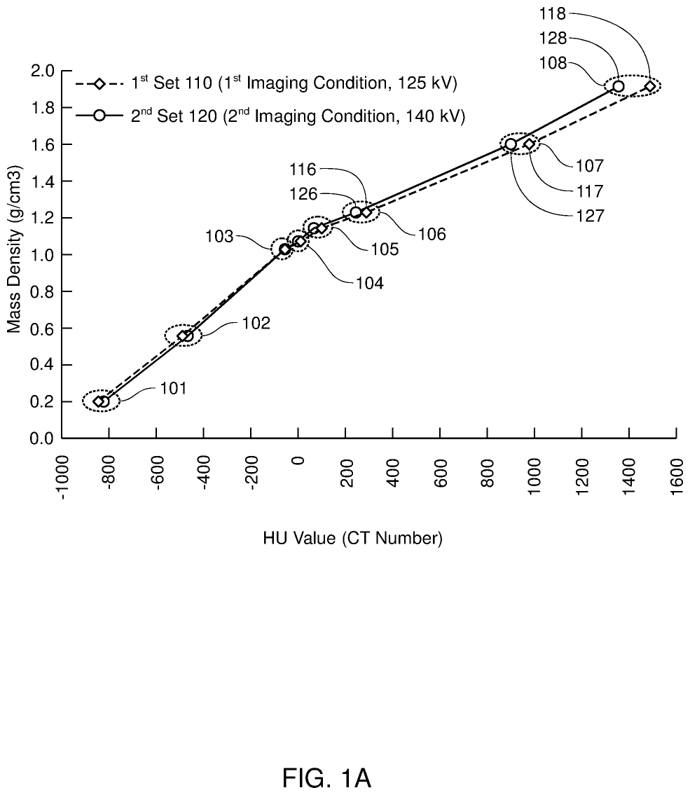

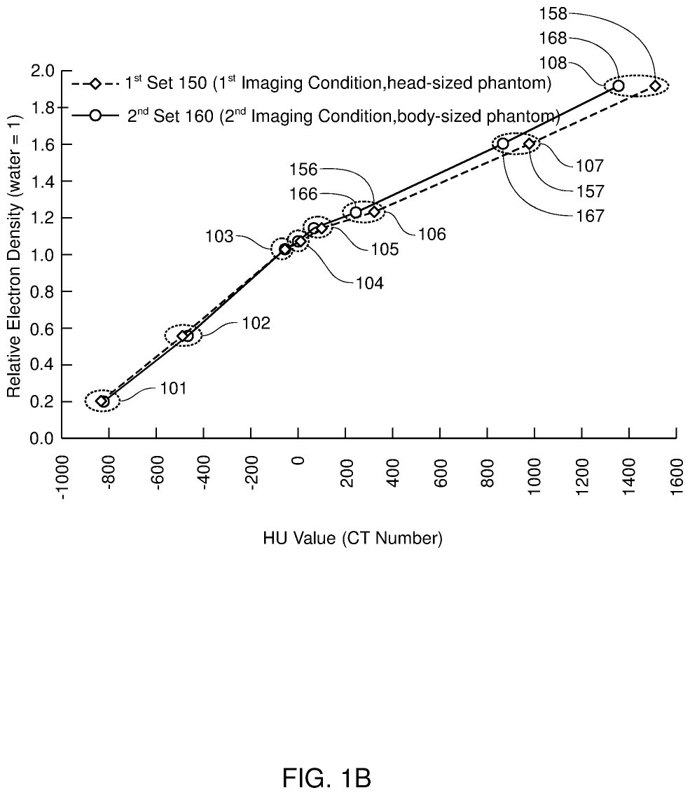

As noted previously, in conventional cone-beam computed tomography (CBCT) reconstructors, there are known systematic limitations in Housfield Unit (HU) accuracy. For example, in typical CBCT imaging systems used in radiotherapy, HU accuracy is commonly stated to be on the order of a ±50 HU difference between a measured and a nominal HU value for a particular phantom material, such as air, Teflon, bone-mimicking materials, or other tissue-mimicking materials. However, such tolerances are usually met when the measured HU value is determined under specific imaging conditions and using a specific imaging phantom and phantom material inserts. For example, when different phantom sizes and/or imaging energies are employed to calibrate a CBCT imaging system, HU accuracy can degrade and a greater difference is observed between a measured and a nominal HU value for a particular phantom material during system calibration. This is because, in conventional reconstructors, HU calibration is obtained by linearly fitting attenuation values measured in various phantom materials to fixed reference HU values. These reference HU values are based on typical manufacturer-provided values (provided, for example, in a Catphan® reference manual) and introduce systematic errors under different imaging conditions. For example, measured HU values can significantly diverge from the reference HU values for a particular phantom material when scanned at different imaging energies, when an imaging X-ray beam undergoes different levels of filtration, and/or when the phantom material is included in a larger or smaller imaging phantom. This is because the measured HU values for a particular material can vary under each of these different imaging conditions, thereby preventing the accurate mapping of a measured HU value in a reconstructed digital volume to a specific value of a radiodensity characteristic, such as electron density or mass density. This variation of measured HU values under different imaging conditions is described below in conjunction with FIGS. 1A and 1B.

FIG. 1A is a graph illustrating mass density as a function of measured HU value. In the instance depicted in FIG. 1A, mass density for eight different phantom materials 101-107 is mapped against two sets of measured HU values (also referred to as CT numbers): a first set 110 and a second set 120. First set 110 includes eight measured HU values, where each of the measured HU values in first set 110 corresponds to a different phantom material that is scanned by a particular CBCT imaging system under a first imaging condition. Similarly, second set 120 includes eight measured HU values, where each of the measured HU values in second set 120 corresponds to the same phantom materials that are scanned by the same CBCT imaging system but under a second imaging condition. Thus, measured HU value 118 in first set 110 and measured HU value 128 in second set 120 correspond to a two different scans of phantom material 108, measured HU value 117 in first set 110 and measured HU value 127 in second set 120 correspond to a two different scans of phantom material 107, measured HU value 116 in first set 110 and measured HU value 126 in second set 120 correspond to a two different scans of phantom material 106, and so on. In the instance illustrated in FIG. 1A, the first imaging condition is substantially identical to the second imaging condition, except for imaging energy. Specifically, in the first imaging condition and the second imaging condition, the same CBCT imaging system is employed to scan an identical imaging phantom, but the scanning energy in the first imaging condition is 125 kV and the scanning energy in the second imaging condition is 140 kV. In other instances, the first imaging condition can vary from the second imaging condition in different and/or additional ways, such as phantom size and/or filtration spectrum.

Each of phantom materials 101-107 has a known mass density value (y-axis) that is associated with a reference HU value (not shown). The reference HU value is typically a manufacturer-provided HU value that is measured under certain imaging conditions and is associated with a specific value of one or more radiodensity characteristics (such as mass density or electron density). Such imaging conditions may not be equivalent to the first imaging condition used to generate measured HU values 111-117 or the second imaging condition used to generate measured HU values 121-127. Therefore, for a particular material, a measured HU value can differ from the reference HU value for that particular material. For example, in the instance illustrated in FIG. 1A, measured HU value 117, which is generated from scanning phantom material 107 under the first imaging condition, differs from measured HU value 127, which is generated from scanning phantom material 107 under the second imaging condition. As a result, when measured HU value 117 or 127 is employed for calculating mass density of patient anatomy, systemic error generally occurs, resulting in inaccurate dose calculations. Further, in many instances, manufacturer-provided reference HU values that can be used to calibrate the measured HU values in first set 110 and the measured HU values in second set 120 are not adapted for the particular CBCT imaging system used to generate the measured HU values of first set 110 or second set 120. This generally also contributes to systemic error in HU values. For example, hardware effects such as off-focal radiation, bowtie scatter, and differences in filtration spectrum can reduce the contrast of phantom materials, leading to attenuation under/over-estimation for some phantom materials. In addition, scatter underestimation can reduce the measured HU values of high-density phantom materials. Some or all of these factors can contribute to measured HU values for phantom materials varying from the manufacturer-provided reference HU values that are associated with a known radiodensity.

FIG. 1A illustrates that a phantom material with a known mass density can have different measured HU values under different imaging conditions. Similarly, FIG. 1B illustrates that a phantom material with a known electron density can have different measured HU values under different imaging conditions. FIG. 1B is a graph illustrating electron density as a function of measured HU value. In the instance depicted in FIG. 1B, electron density for eight different phantom materials 101-108 is mapped against two sets of measured HU values: a first set 150 and a second set 160. First set 150 includes measured HU values that each correspond to a different phantom material imaged by a particular CBCT imaging system under a first imaging condition. Similarly, second set 160 includes measured HU values that each correspond to a different phantom material imaged by the same CBCT imaging system but under a second imaging condition. In the instance illustrated in FIG. 1B, the first imaging condition is substantially identical to the second imaging condition, except for phantom size. Specifically, in the first imaging condition and the second imaging condition, the same CBCT imaging system is employed to scan using the same scanning energy (e.g., 125 kV), but the size of the imaging phantom in the first imaging condition is a head-sized phantom and the size of the imaging phantom in the second imaging condition is a body-sized phantom.

As shown in the instance illustrated in FIG. 1B, the measured HU values 156-158, which are generated from scanning phantom materials 106-108 under the first imaging condition, differ significantly from measured HU values 166-168, which are generated from scanning phantom materials 106-108 under the second imaging condition. As a result, when measured HU values 156-158 or 166-168 are employed for calculating mass density of patient anatomy, systemic error generally occurs, resulting in inaccurate dose calculations.

In light of the above, there is a need in the art for improved techniques for calibrating attenuation values from CBCT imaging.

According to various embodiments, measured HU values generated via CBCT imaging are mapped with higher accuracy to specific values of radiodensity, such as an electron density value or a mass density value. Specifically, in the embodiments, a plurality of system-specific calibration curves are generated for a particular CBCT imaging system, where each calibration curve is associated with a different imaging condition of the CBCT imaging system. When a digital volume is reconstructed based on projection images generated by the CBCT imaging system with a particular imaging condition, HU values of the digital volume are modified using the calibration curve associated with that particular imaging condition. Consequently, the modified HU values are more accurately mapped to physical characteristics of different materials, such as mass density or electron density, thereby enabling more accurate determination of dosing. Various example embodiments are described below.

### System Overview

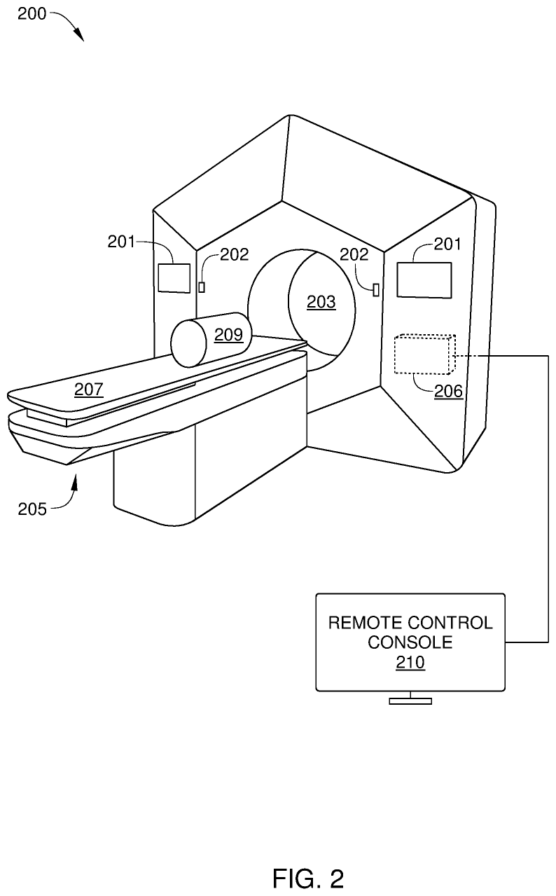

FIG. 2 is a perspective view of a radiation therapy system 200 that can beneficially implement various embodiments. In some embodiments, radiation therapy system 200 includes an imaging system configured to image patient anatomy using X-ray imaging techniques. For example, in some embodiments, radiation therapy system 200 is configured to provide stereotactic radiosurgery and/or precision radiotherapy for lesions, tumors, and conditions anywhere in the body where radiation treatment is indicated. As such, radiation therapy system 200 can include one or more of a linear accelerator (LINAC) that generates a megavolt (MV) treatment beam of high energy X-rays, one or more kilovolt (kV) X-ray sources, one or more X-ray imagers, and, in some embodiments, an MV electronic portal imaging device (EPID). By way of example, radiation therapy system 200 is described herein configured with a circular gantry. In other embodiments, radiation therapy system 200 can be configured with a C-gantry capable of infinite rotation via a slip ring connection. In yet other embodiments, radiation therapy system 200 can be configured with an imaging system having an MRI-based imaging capability.

In some embodiments, radiation therapy system 200 is capable of kV imaging of a target volume immediately prior to or during application of an MV treatment beam, so that an IGRT and/or an intensity-modulated radiation therapy (IMRT) process can be performed using X-ray imaging. Radiation therapy system 200 may include one or more touchscreens 201, couch motion controls 202, a bore 203, a base positioning assembly 205, a couch 207 disposed on base positioning assembly 205, and an image acquisition and treatment control computer 206, all of which are disposed within a treatment room. Radiation therapy system 200 further includes a remote control console 210, which is disposed outside the treatment room and enables treatment delivery and patient monitoring from a remote location. Base positioning assembly 205 is configured to precisely position couch 207 with respect to bore 203, and motion controls 202 include input devices, such as button and/or switches, that enable a user to operate base positioning assembly 205 to automatically and precisely position couch 207 to a predetermined location with respect to bore 203. Motion controls 202 also enable a user to manually position couch 207 to a predetermined location. Also shown in FIG. 2 is an imaging phantom 209 disposed on couch 207, for example in preparation for the performance of a calibration process on radiation therapy system 200.

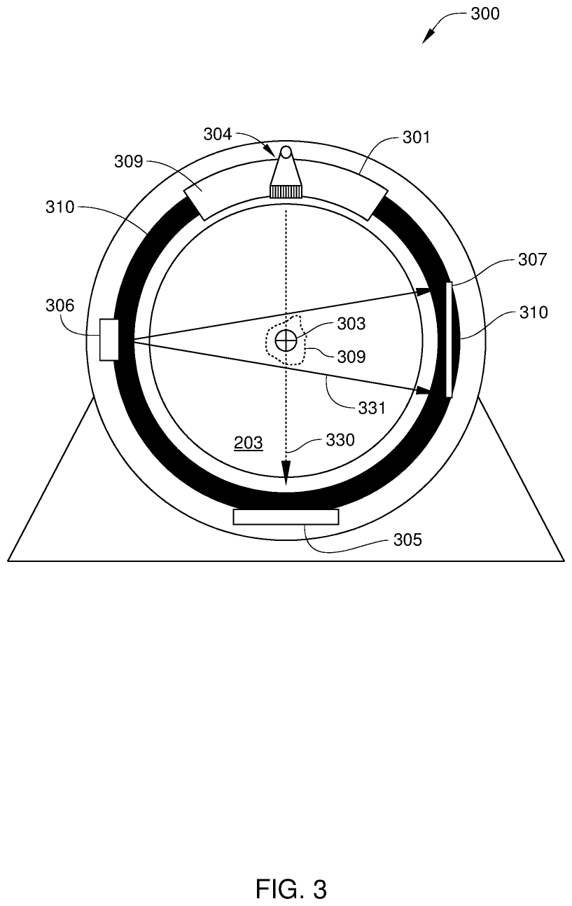

FIG. 3 schematically illustrates a drive stand 300 and a gantry 310 of radiation therapy system 200, according to various embodiments. Covers, base positioning assembly 205, couch 207, and other components of radiation therapy system 200 are omitted in FIG. 3 for clarity. Drive stand 300 is a fixed support structure for components of radiation therapy system 200, including gantry 310 and a drive system 301 for rotatably moving gantry 310. Drive stand 300 rests on and/or is fixed to a support surface that is external to radiation therapy system 200, such as a floor of a radiotherapy treatment facility. Gantry 310 is rotationally coupled to drive stand 300 and is a support structure on which various components of radiation therapy system 200 are mounted, including a linear accelerator (LINAC) 304, an MV electronic portal imaging device (EPID) 305, an imaging X-ray source 306, and an X-ray imager 307. During operation of radiation therapy system 200, gantry 320 rotates about bore 203 when actuated by drive system 301.

Drive system 301 rotationally actuates gantry 310. In some embodiments, drive system 301 includes a linear motor that can be fixed to drive stand 300 and interacts with a magnetic track (not shown) mounted on gantry 310. In other embodiments, drive system 301 includes another suitable drive mechanism for precisely rotating gantry 310 about bore 301. LINAC 304 generates an MV treatment beam 330 of high energy X-rays (or in some embodiments electrons, protons, and/or other heavy charged particles, ultra-high dose rate X-rays (e.g., for FLASH radiotherapy) or microbeams for microbeam radiation therapy) and EPID 305 is configured to acquire X-ray images with treatment beam 330. Imaging X-ray source 306 is configured to direct a conical beam of X-rays, referred to herein as imaging X-rays 331, through an isocenter 303 of radiation therapy system 200 to X-ray imager 307, and isocenter 303 typically corresponds to the location of a target volume 309 to be treated. In the embodiment illustrated in FIG. 3, X-ray imager 307 is depicted as a planar device, whereas in other embodiments, X-ray imager 307 can have a curved configuration. Imaging X-ray source 306 and X-ray imager 307 can be employed as imaging device 131 in FIG. 1.

X-ray imager 307 receives imaging X-rays 331 and generates suitable projection images therefrom. According to certain embodiments, such projection images can then be employed to construct or update portions of imaging data for a digital volume that corresponds to a three-dimensional (3D) region that includes target volume 309. That is, a 3D image of such a 3D region is reconstructed from the projection images. In some embodiments, the 3D image is reconstructed from projection images that are acquired with couch 207 in FIG. 2 located at different imaging positions. In some embodiments, CBCT and/or digital tomosynthesis (DTS) can be used to process the projection images generated by X-ray imager 307. CBCT is often employed at the beginning of a radiation therapy session to generate a set-up 3D reconstruction. For example, CBCT may be employed immediately prior to application of treatment beam 330 to generate a 3D reconstruction confirming that target volume 309 has not moved or changed shape.

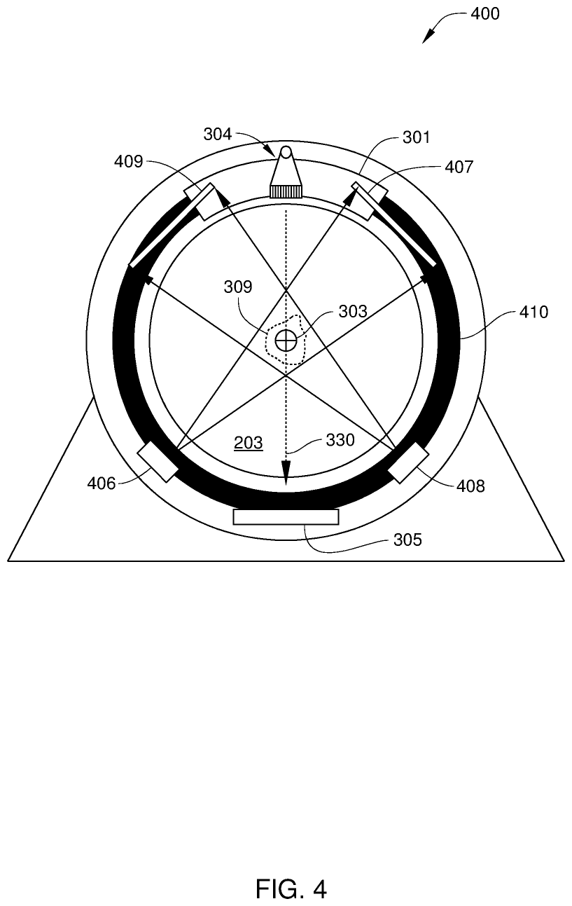

In the embodiment illustrated in FIG. 3, radiation therapy system 200 includes a single X-ray imager and a single corresponding imaging X-ray source. In other embodiments, radiation therapy system 200 can include two or more X-ray imagers, each with a corresponding imaging X-ray source. One such embodiment is illustrated in FIG. 4.

FIG. 4 schematically illustrates a drive stand 400 and gantry 410 of radiation therapy system 200, according to various embodiments. Drive stand 400 and gantry 410 are substantially similar in configuration to drive stand 300 and gantry 300 in FIG. 3, except that the components of radiation therapy system 200 that are mounted on gantry 410 include a first imaging X-ray source 406, a first X-ray imager 407, a second imaging X-ray source 408, and a second X-ray imager 409. In such embodiments, the inclusion of multiple X-ray imagers in radiation therapy system 200 facilitates the generation of projection images (for reconstructing the target volume) over a shorter image acquisition arc. For instance, in some instances, when radiation therapy system 200 includes two X-ray imagers and corresponding X-ray sources, sufficient information can be collected to reconstruct a 3D region that includes target volume 409 without rotating gantry 410. Alternatively, in some instances, when radiation therapy system 200 includes two X-ray imagers and corresponding X-ray sources, an image acquisition arc for acquiring projection images of a certain image quality can be approximately half that for acquiring projection images of a similar image quality with a single X-ray imager and X-ray source.

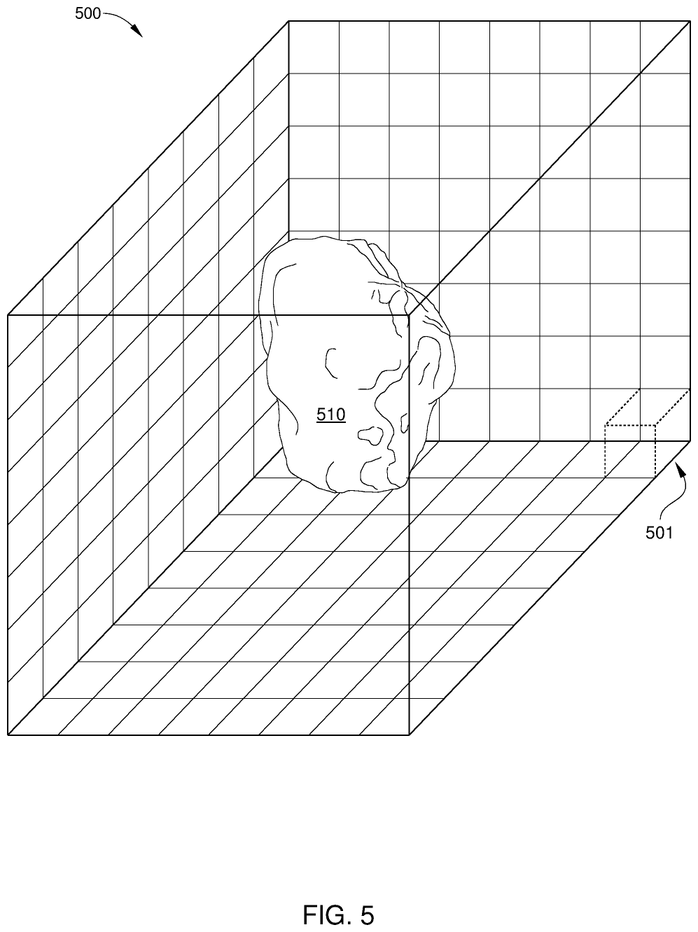

The projection images generated by X-ray imager 307 in FIG. 3 (or by first x-ray imager 407 and second X-ray imager 409 in FIG. 4) are used to reconstruct a 3D digital volume of an object or portion of patient anatomy, such as a 3D region of patient anatomy that includes target volume 309. One embodiment of such a digital volume is described below in conjunction with FIG. 5.

FIG. 5 schematically illustrates a digital volume 500 that is constructed based on projection images generated by one or more X-ray imagers included in radiation therapy system 200, according to various embodiments. For example, in some embodiments, the projection images can be generated by a single X-ray imager, such as X-ray imager 307, and in other embodiments the projection images can be generated by multiple X-ray imagers, such as first x-ray imager 407 and second X-ray imager 409.

Digital volume 500 includes a plurality of voxels 501 (dashed lines) of anatomical image data, where each voxel 501 corresponds to a different location within digital volume 500. For clarity, only a single voxel 501 is shown in FIG. 5. Digital volume 500 corresponds to a 3D region that includes target volume 510. In FIG. 5, digital volume 500 is depicted as an 8×8×8 voxel cube, but in practice, digital volume 500 generally includes many more voxels, for example orders of magnitude more than are shown in FIG. 5. Generally, a different HU value is associated with each voxel 501.

For purposes of discussion, target volume 510 can refer to the gross tumor volume (GTV), clinical target volume (CTV), or the planning target volume (PTV) for a particular treatment. The GTV depicts the position and extent of the gross tumor, for example what can be seen or imaged; the CTV includes the GTV and an additional margin for sub-clinical disease spread, which is generally not imageable; and the PTV is a geometric concept designed to ensure that a suitable radiotherapy dose is actually delivered to the CTV without adversely affecting nearby organs at risk. Thus, the PTV is generally larger than the CTV, but in some situations can also be reduced in some portions to provide a safety margin around an organ at risk. The PTV is typically determined based on imaging performed prior to the time of treatment, and alignment of the PTV with the current position of patient anatomy at the time of treatment is facilitated by X-ray imaging of digital volume 500.

In some embodiments, image information associated with each voxel 501 of digital volume 500 is constructed via projection images generated by the single or multiple X-ray imagers of radiation therapy system 200 via a CBCT process. For example, such a CBCT process can be employed immediately prior to delivering treatment beam 330 (shown in FIG. 3) to target volume 510, so that the location and shape of target volume 510 can be confirmed before treatment begins.

### Generation of Attenuation Calibration Curves

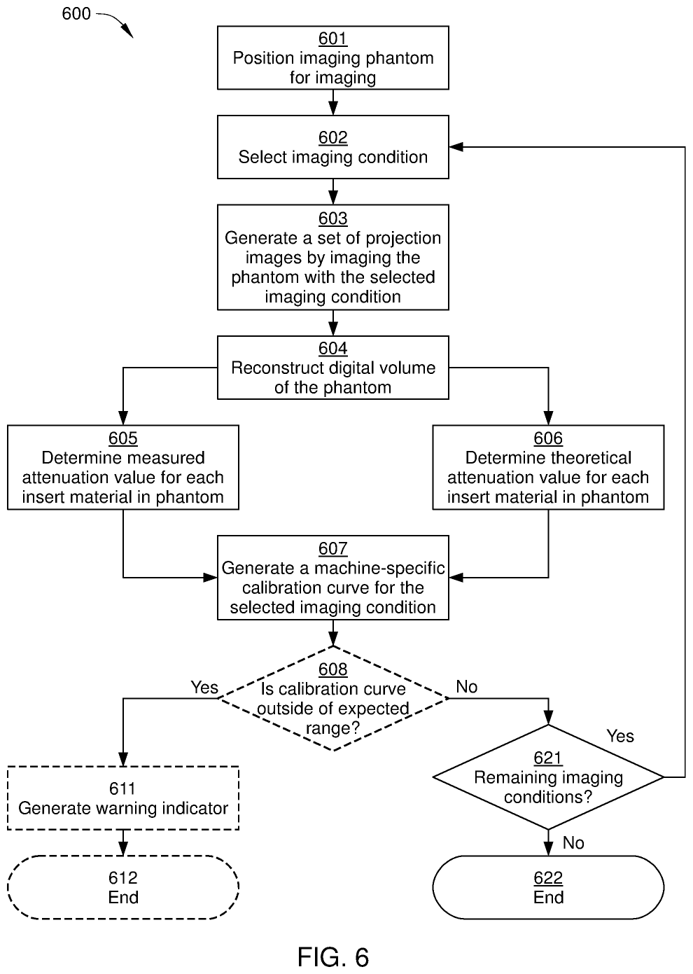

According to various embodiments, dosing of target volume 510 and/or other portions of digital volume 500 can be determined based on the HU values associated with each voxel 501. In such embodiments, the HU values associated with each voxel 501 of digital volume 500 are modified using a calibration curve that corresponds with the particular imaging conditions employed to reconstruct digital volume 500. The modified HU values indicate an accurate attenuation value for each voxel 501, and are mapped to specific values of a physical characteristic (such as mass density or electron density). Based on the values of the physical characteristic and on treatment beam information, accurate dosing of target volume 510 and/or other portions of digital volume can be calculated or simulated. Generation of calibration curves that correspond with particular imaging conditions is described below in conjunction with FIG. 6.

FIG. 6 sets forth a flowchart of a computer-implemented method 600 for a radiotherapy system, according to one or more embodiments. In some embodiments, computer-implemented method 600 can be performed as part of a system calibration process and/or a periodic system testing process for radiation therapy system 200. Computer-implemented method 600 may include one or more operations, functions, or actions as illustrated by one or more of blocks 602-622. Although the blocks are illustrated in a sequential order, these blocks may be performed in parallel, and/or in a different order than those described herein. Also, the various blocks may be combined into fewer blocks, divided into additional blocks, and/or eliminated based upon the desired implementation. Although computer-implemented method 600 is described in conjunction with radiation therapy system 200 and FIGS. 2-5, persons skilled in the art will understand that performance of computer-implemented method 600 by any suitably configured radiation therapy system is within the scope of the present embodiments.

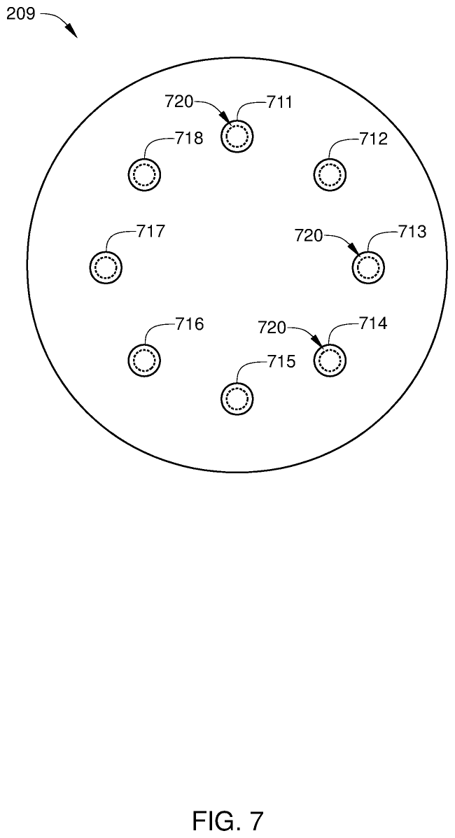

Computer-implemented method 600 begins at step 601, where an imaging phantom 209 is positioned for imaging by an imaging system associated with radiation therapy system 200, such as imaging X-ray source 306 and X-ray imager 307 or LINAC 304 and EPID 305. For example, imaging phantom 209 may be disposed on couch 207 of radiation therapy system 200. Generally, imaging phantom 209 includes a plurality of material inserts, as shown in FIG. 7.

FIG. 7 schematically illustrates an example embodiment of imaging phantom 209. As shown in the embodiment depicted in FIG. 7, imaging phantom includes a plurality of material inserts 711-718 arranged within imaging phantom 209. Each material insert 711-718 can include a material having a different but known radiodensity value. Generally, material inserts 711-718 are selected to include materials that have a range of different radio density values. Thus, HU values generated by an imaging system associated with radiation therapy system 200 can be calibrated for most or all conditions encountered in clinical use of radiation therapy system 200. Examples of such materials include, without limitation, air, polymethylpentene (PMP), low-density polyethylene (LDPE), polystyrene, acrylic, Delrin®, Teflon®, 50% bone-mimicking materials, 20% bone-mimicking materials, liver tissue-mimicking materials, breast tissue-mimicking materials, other tissue-mimicking materials, and the like. In the embodiment illustrated in FIG. 7, imaging phantom 209 includes eight material inserts, but in other embodiments, imaging phantom 209 can include more than or fewer than eight material inserts, and/or duplicate material inserts.

As shown, each material insert 711-718 includes a non-edge region 720 (dashed lines) that is disposed within the material insert and excludes any surfaces of the material insert. In some embodiments, HU values are determined for material inserts 711-718 based on non-edge regions 720 to avoid or reduce systemic errors caused by border effects.

Returning to FIG. 6, in step 602, a specific imaging condition is selected. In some embodiments, the imaging condition includes a particular combination of factors that can affect the HU values generated by an imaging system associated with radiation therapy system 200. For example, in some embodiments, the imaging condition includes a specific imaging energy (e.g., 120 kV, 140 kV, etc.). As described above in conjunction with FIG. 1A, there is a known dependency of HU values of an imaged material on the scanning energy employed to image the material. Alternatively or additionally, in some embodiments, the imaging condition includes a size of phantom 209. As described above in conjunction with FIG. 1B, there is a known dependency of HU values of an imaged material on the size of the phantom (or region of patient anatomy) being imagined. This effect is attributed to beam hardening associated with larger phantoms or patient anatomies. Alternatively or additionally, in some embodiments, the imaging condition includes a specific filtration spectrum associated with the imaging system. Thus, in some embodiments, the specific imaging condition selected may include a particular scanning energy, a particular size of the phantom (or region of patient anatomy) being imagined, and/or a specific X-ray filtration spectrum employed during the imaging of the phantom (or region of patient anatomy).

In step 603, a set of projection images is generated by imaging or scanning imaging phantom 209 with the imaging condition selected in step 602. For example, in some embodiments, the set of projection images is generated via a CBCT process with imaging X-ray source 306 and X-ray imager 307.

In step 604, a digital volume of imaging phantom 209 is reconstructed based on the projection images generated in step 603. In some embodiments, the reconstruction process employed in step 604 includes a spectral model for imaging X-ray source 306 and/or a beam-hardening correction that contemplates phantom (or patient anatomy) size, and accounts for the effects of a detector efficiency of X-ray imager 307 and/or the specific filtration spectrum associated with imaging X-ray source 306.

In step 605, a measured attenuation value, such as an HU value, is determined for each material insert 711-718 of imaging phantom 209. In some embodiments, an HU value is determined for a particular material insert 711-718 by selecting a region of the digital volume reconstructed in step 604 that corresponds to that particular material insert. Typically, such a region consists of a plurality of voxels 501, and determining the HU value for that particular material insert is based on the HU values associated with some or all of this plurality of voxels 501. For example, in some embodiments, the HU value for that particular material insert is determined as an average HU value of some or all of this plurality of voxels 501. Additionally or alternatively, in some embodiments, to avoid or reduce systemic errors caused by border effects, the HU value for a particular material insert is based on the HU values associated with a non-edge portion 720 of that particular material insert.

In step 606, a theoretical attenuation value, such as an HU value, is determined for each material insert 711-718 of imaging phantom 209. In the embodiment illustrated in FIG. 6, step 606 is performed as part of computer-implemented method 600. In other embodiments, theoretical attenuation values for material inserts are determined prior to implementation of computer-implemented method 600. In some embodiments, each theoretical HU value for material inserts 711-718 is determined based on some or all of the following inputs: mass density and chemical composition of the material, spectrum of imaging X-ray source 306, detector efficiency of X-ray imager 307, and average thickness or beam-hardening expected by imaging phantom 209 (or the region of patient anatomy being imaged).

In some embodiments, based on mass density and chemical composition of a particular material, the attenuation coefficients of this material can be calculated (for example, based on the published NIST data). In some embodiments, the attenuation coefficient spectra are calculated in 1 kV-steps (for example, for kV-CBCT up to a maximum energy of 140 kV).

In some embodiments, the spectrum of the X-ray source includes some or all of the filtration components used in a particular radiotherapy system (e.g., inherent filtration of a kV tube included in imaging X-ray source 306, additional filters such as a Titanium filter, filtration introduced by beam-shaping components such as bowtie filters, and/or the like). In some embodiments, a raw spectrum of the kV-tube anode can be obtained from Monte Carlo simulations, with additional filtration applied on top of this.

In some embodiments, detector efficiency represents the percentage of deposited energy for a given energy value. In such embodiments, detector efficiency is included to calculate the signal that X-ray imager 307 actually sees. Thus, in practice, this means that higher energies generally contribute relatively less to the signal. In some embodiments, the detector efficiency can be calculated from Monte Carlo simulations of the complete detector stack-up based on the specific configuration and design of the detector included in radiotherapy system 200. Alternatively, in some embodiments, when a thickness of the scintillator material included in the detector is known, the detector efficiency is estimated from publicly available energy absorption spectra of the scintillator material.

In some embodiments, average thickness or beam-hardening expected by imaging phantom 209 (or the imaged region of patient anatomy) is taken into account to get the apparent HU value of a material. Thus, in such embodiments, the phantom size is taken into account to get the apparent HU value of each material insert 711-718 of imaging phantom 209. In particular, in such embodiments, the HU value of a non-water material is not a fixed value given by the material and source energy, and instead depends on the size of the patient or of imaging phantom 209. In some embodiments, two size cases are employed: an instance with a hardening of 20 cm water diameter and 5 mm Aluminum, (representing typical head-size scan protocols as well as protocols for HU calibration phantom scans) and an instance with 30 cm water and 10 mm Aluminum (more representative of typical body-size scan protocols). In other embodiments, beam-hardening can be determined for many more size cases.

In some embodiments, for the calculation of the theoretically expected HU values in step 606, these values are defined by the apparent material attenuation relative to the attenuation of water under the same condition. These material attenuations are determined in step 606 from the theoretically expected detector signal by integrating the contributions over all X-ray photons energies and applying all the factors given above. In such embodiments, in this integral, the contribution of a given energy is proportional to the amount of photons, the energy, and the detection efficiency. In such embodiments, an energy-integrating detector can be assumed.

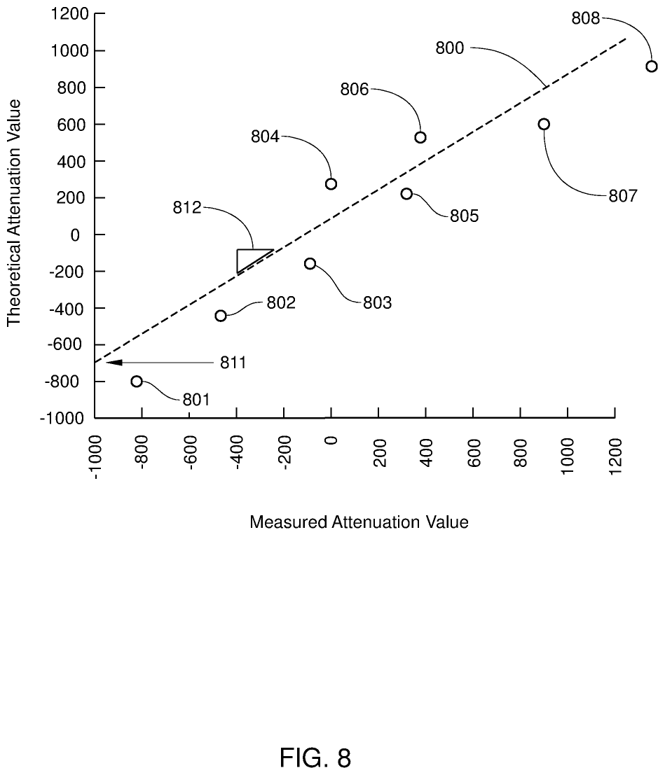

In step 607, a machine-specific calibration curve is generated for the imaging condition selected in step 602. In some embodiments, the machine-specific calibration curve is generated based on measured attenuation (HU) values determined in step 605 and on corresponding theoretical attenuation values determined in step 606. For example, in some embodiments, the calibration curve for the selected imaging condition includes a linear curve with a slope value and an intercept value. Embodiments of such calibration curves are described below in conjunction with FIG. 8.

FIG. 8 illustrates a calibration curve 800, according to various embodiments. Calibration curve 800 is constructed based on measured attenuation values for a plurality of material inserts 801-808 and on corresponding theoretical attenuation values determined for the same material inserts 801-808. As shown, calibration curve 800 is generated by plotting, for each material insert, the measured attenuation value for that material insert vs. the corresponding theoretical attenuation value for that material insert. In the embodiment illustrated in FIG. 8, calibration curve 800 includes a linear curve that is determined, for example, using a least-square fit. Thus, in the embodiment illustrated in FIG. 8, calibration curve 800 has the characteristics of a slope value 811 and an intercept value 812 that are associated with the linear curve. In embodiments in which calibration curve 800 includes a non-linear portion, calibration curve 800 can have one or more characteristics that quantitatively describe the non-linear portion of calibration curve 800, such as values for exponents, coefficients, and the like.

Returning to FIG. 6, in optional step 608, radiation therapy system 200 determines whether the calibration curve is outside of an expected range from a theoretical calibration curve. In some embodiments, in step 608, radiation therapy system 200 determines whether one or more characteristics of the calibration curve generated in step 607 differ from a theoretical value for the corresponding characteristic(s) of such a theoretical calibration curve. For example, in some embodiments, radiation therapy system 200 determines whether one or more characteristics of the calibration curve generated in step 607 (such as a slope value and/or an intercept value) differs from one or more corresponding characteristics of a theoretical calibration curve that indicates a theoretically expected relationship between the measured attenuation values and corresponding theoretical attenuation values under specified imaging conditions. When the difference between the one or more characteristics of the calibration curve generated in step 607 and the one or more corresponding characteristics of theoretical calibration curve exceeds a threshold value, computer-implemented method 600 proceeds to step 611. When such a difference does not exceed the threshold value, computer-implemented method 600 proceeds to step 621.

Optional step 611 is performed in response to the calibration curve generated in step 607 being outside of an expected range. In optional step 611, radiation therapy system 100 generates a warning indicator, such as an output displayed by remote control console 210. Computer-implemented method 600 then proceeds to step 612 and ends. Thus, in some embodiments, optional steps 608, 611, and 612 can be employed as a system consistency check that can detect certain failure modes or sources of error. For example, in some embodiments, a large deviation of the calibration curve for specific imaging conditions from a theoretical calibration curve associated with the same imaging conditions can identify technical issues associated with a particular radiotherapy system, such as miscalculation of one or more subsystems of radiation therapy system 200.

In step 621, radiation therapy system 200 determines whether there are any remaining imaging conditions for which a calibration curve is to be generated. In some embodiments, different imaging conditions are based on a relatively coarse set of different imaging energies (e.g., 80 kV, 100 kV, 125 kV, 140 kV). Similarly, in some embodiments, different imaging conditions are based on patient-caused beam hardening for a relatively coarse set of phantom/patient anatomy sizes, such as a head-sized scan protocol and a body-sized scan protocol. Thus, in an embodiment in which imaging conditions are based on four different imaging energies and two phantom/patient anatomy sizes, there are a total of eight calibration curves to be generated. Further, when additional factors are considered, such as the filtration spectrum of imaging X-ray source 306, the total number of calibration curves to be generated increases proportionately. When the determination is made that there are remaining imaging conditions for which a calibration curve is to be generated, computer-implemented method 600 returns to step 602. When the determination is made that there are no remaining imaging conditions for which a calibration curve is to be generated, computer-implemented method 600 proceeds to step 622 and ends.

### Determination of X-Ray Dose Using Attenuation Calibration Curves

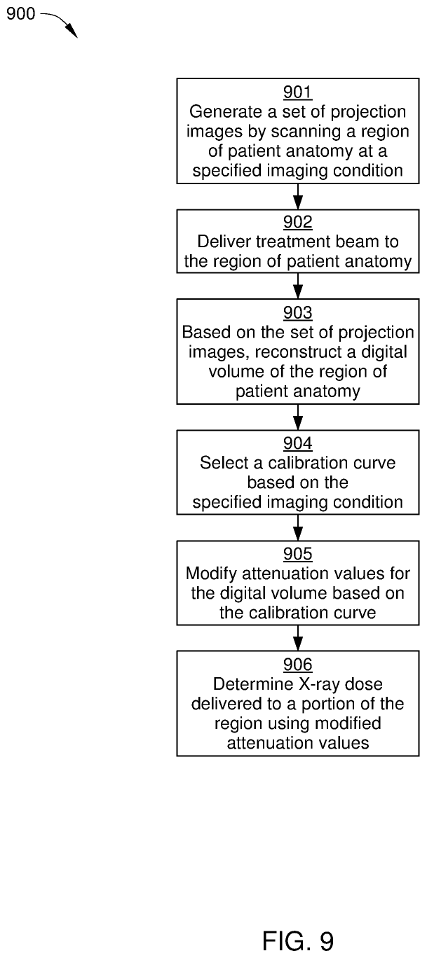

According to various embodiments, dosing of a target volume and/or other portions of a digital volume can be determined based CBCT HU values of the digital volume and on the calibration curves generated via computer-implemented method 600 of FIG. 6. One example embodiment is described below in conjunction with FIG. 9.

FIG. 9 sets forth a flowchart of a computer-implemented method 900 for a radiotherapy system, according to one or more embodiments. In some embodiments, computer-implemented method 900 can be performed as part of a treatment fraction. Alternatively or additionally, in some embodiments, computer-implemented method 900 can be performed prior to delivery of a treatment fraction as part of a simulation and/or treatment planning process. In such embodiments, delivery of the treatment beam is a simulated treatment beam.

Computer-implemented method 900 may include one or more operations, functions, or actions as illustrated by one or more of blocks 901-906. Although the blocks are illustrated in a sequential order, these blocks may be performed in parallel, and/or in a different order than those described herein. Also, the various blocks may be combined into fewer blocks, divided into additional blocks, and/or eliminated based upon the desired implementation. Although computer-implemented method 900 is described in conjunction with radiation therapy system 200 and FIGS. 2-5, persons skilled in the art will understand that performance of computer-implemented method 900 by any suitably configured radiation therapy system is within the scope of the present embodiments.

Prior to computer-implemented method 900, a patient is positioned for treatment on couch 207 for treatment. In addition, radiation therapy system 200 is configured to perform imaging and/or treatment associated with a particular treatment fraction for the patient.

Computer-implemented method 900 begins at step 901, where a set of projection images are generated by scanning a region of patient anatomy at a specified imaging condition. For example, in some embodiments, the projection images are generated via a CBCT imaging process using an imaging system associated with radiation therapy system 200, such as imaging X-ray source 306 and X-ray imager 307 and/or LINAC 304 and EPID 305. In some embodiments, the specified imaging condition can include a particular imaging energy and a particular size of patient anatomy (e.g., head or torso). In some embodiments, the specified imaging condition further includes a particular filtration spectrum and/or some other factor that affects the accuracy of HU values in CBCT imaging.

In step 902, treatment beam 330 is delivered to the region of patient anatomy. In embodiments in which computer-implemented method 900 is performed as part of a simulation and/or a treatment planning process, treatment beam 330 is a simulation of an actual treatment beam.

In step 903, based on the set of projection images generated in step 901, a digital volume of the region of patient anatomy of interest is reconstructed. Similar to the reconstruction process in step 604 of computer-implemented method 600, in some embodiments, the reconstruction process employed in step 903 includes a spectral model for imaging X-ray source 306 and/or a beam-hardening correction that contemplates phantom (or patient anatomy) size, and accounts for the effects of a detector efficiency of X-ray imager 307 and/or the specific filtration spectrum associated with imaging X-ray source 306.

In step 904, a calibration curve is selected based on the specified imaging condition. In step 905, attenuation values (e.g., HU values) for the digital volume reconstructed in step 903 are modified based on the selected calibration curve. Thus, given a particular HU value for a specific voxel 501 of the digital volume, the selected calibration curve indicates a modified value. In the embodiment described in conjunction with FIG. 9, the modification of attenuation values in step 904 is described in terms of determining a modified value by referencing a selected calibration curve. In other embodiments, the selected calibration curve can be implemented in any other technically feasible way, for example as a lookup table, a mathematical expression, and the like.

In step 906, an X-ray dose delivered to some or all portions of the digital volume is determined based on the modified attenuation values generated in step 904. For example, in some embodiments, the X-ray dose delivered to a particular region of the digital volume is determined by first selecting, for each voxel of the digital volume, a value for a physical characteristic (such as mass density or electron density) that corresponds to the modified attenuation value for that voxel. A conventional radiation dose calculation is then performed for some or all portions of the digital volume using the values for the physical characteristic.

### Example Computing Device

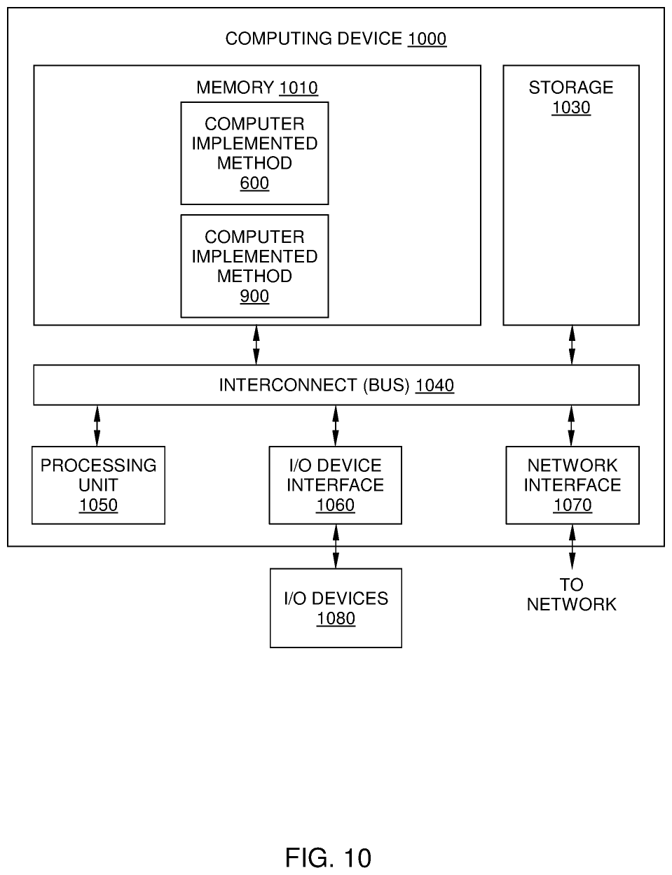

FIG. 10 is an illustration of computing device 1000 configured to perform various embodiments described herein. For example, in some embodiments, computing device 1000 can be implemented as image acquisition and treatment control computer 206 and/or remote control console 210 in FIG. 2. Computing device 1000 may be a desktop computer, a laptop computer, a smart phone, or any other type of computing device suitable for practicing one or more embodiments of the present disclosure. In operation, computing device 1000 is configured to execute instructions associated with computer-implemented method 600 and/or computer-implemented method 900, as described herein. It is noted that the computing device described herein is illustrative and that any other technically feasible configurations fall within the scope of the present disclosure.

As shown, computing device 1000 includes, without limitation, an interconnect (bus) 1040 that connects a processing unit 1050, an input/output (I/O) device interface 1060 coupled to input/output (I/O) devices 1080, memory 1010, a storage 1030, and a network interface 1070. Processing unit 1050 may be any suitable processor implemented as a central processing unit (CPU), a graphics processing unit (GPU), an application-specific integrated circuit (ASIC), a field programmable gate array (FPGA), any other type of processing unit, or a combination of different processing units, such as a CPU configured to operate in conjunction with a GPU or digital signal processor (DSP). In general, processing unit 1050 may be any technically feasible hardware unit capable of processing data and/or executing software applications, including computer-implemented method 600 and/or computer-implemented method 900.

I/O devices 1080 may include devices capable of providing input, such as a keyboard, a mouse, a touch-sensitive screen, and so forth, as well as devices capable of providing output, such as a display device and the like. Additionally, I/O devices 1080 may include devices capable of both receiving input and providing output, such as a touchscreen, a universal serial bus (USB) port, and so forth. I/O devices 1080 may be configured to receive various types of input from an end-user of computing device 1000, and to also provide various types of output to the end-user of computing device 1000, such as displayed digital images or digital videos. In some embodiments, one or more of I/O devices 1080 are configured to couple computing device 1000 to a network.

Memory 1010 may include a random access memory (RAM) module, a flash memory unit, or any other type of memory unit or combination thereof. Processing unit 1050, I/O device interface 1060, and network interface 1070 are configured to read data from and write data to memory 1010. Memory 1010 includes various software programs that can be executed by processor 1050 and application data associated with said software programs, including computer-implemented method 600 and/or computer-implemented method 900.

### Example Computer Program Product

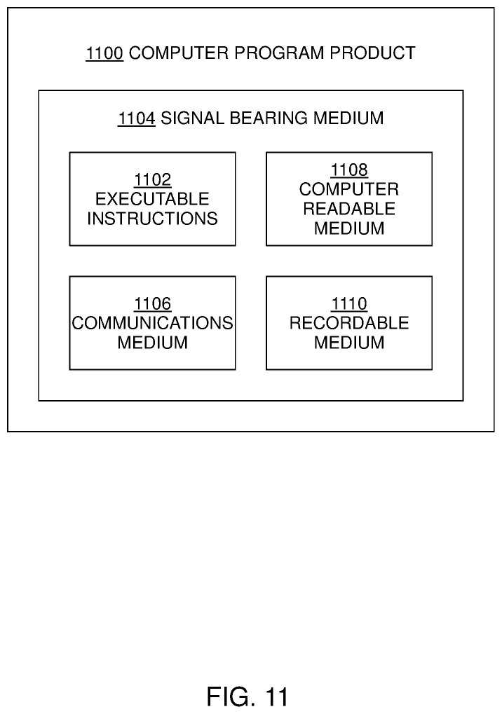

FIG. 11 is a block diagram of an illustrative embodiment of a computer program product 1100 for implementing a method for reducing scatter in an X-ray projection image, according to various embodiments. Computer program product 1100 may include a signal bearing medium 1104. Signal bearing medium 1104 may include one or more sets of executable instructions 1102 that, when executed by, for example, a processor of a computing device, may provide at least the functionality described above with respect to FIGS. 1-9.

In some implementations, signal bearing medium 1104 may encompass a non-transitory computer readable medium 1108, such as, but not limited to, a hard disk drive, a Compact Disc (CD), a Digital Video Disk (DVD), a digital tape, memory, etc. In some implementations, signal bearing medium 1104 may encompass a recordable medium 1110, such as, but not limited to, memory, read/write (R/W) CDs, R/W DVDs, etc. In some implementations, signal bearing medium 1104 may encompass a communications medium 1106, such as, but not limited to, a digital and/or an analog communication medium (e.g., a fiber optic cable, a waveguide, a wired communications link, a wireless communication link, etc.). Computer program product 1100 may be recorded on non-transitory computer readable medium 1108 or another similar recordable medium 1110.

In sum, embodiments described herein enable the higher HU accuracy available from clinical CT scanners to be realized with a CBCT imaging system, such as an on-board CBCT imaging system that is in included in a radiotherapy system. In the embodiments, a plurality of system-specific calibration curves are generated for a particular CBCT imaging system, where each calibration curve is associated with a different imaging condition of the CBCT imaging system. Thus, HU values of a reconstructed volume can be modified using the calibration curve associated with that particular imaging condition. Because the modified HU values are more accurately mapped to physical characteristics of different materials, such as mass density or electron density, the modified HU values can be used to determine more accurate dosing.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. Throughout the present disclosure, the terms “first,” “second,” etc. do not denote any order of importance, but are rather used to distinguish one element from another. Independent of the grammatical term usage, individuals with male, female or other gender identities are included within the term.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A computer-implemented method of determining X-ray dose delivered to a region of patient anatomy, the method comprising:
using a first imaging condition, generating a set of projection images of a region of patient anatomy;
based on the set of projection images of the target volume, reconstructing a digital volume that includes a target volume disposed within the region of patient anatomy;
based on the digital volume, determine current position of the target volume within the region of patient anatomy;
delivering a treatment beam to the target volume while disposed at the current position;
based on the first imaging condition, selecting a first calibration curve from a plurality of calibration curves, wherein each calibration curve in the plurality of calibration curves is associated with a different imaging condition; and
based on the first calibration curve, determining an x-ray dose delivered to a portion of the target volume.

2. The computer-implemented method of claim 1, wherein the first imaging condition comprises a first size of the region of patient anatomy and a first imaging energy.

3. The computer-implemented method of claim 2, wherein the first imaging condition further comprises a first X-ray filtration spectrum.

4. The computer-implemented method of claim 2, wherein the first size of the region of patient anatomy is selected from one of a head-sized region of patient anatomy and a torso-sized region of patient anatomy.

5. The computer-implemented method of claim 1, wherein:
the digital volume includes a plurality of voxels; and
determining the X-ray dose delivered to the portion of the target volume comprises determining, for each voxel in the plurality of voxels, a value for a physical characteristic.

6. The computer-implemented method of claim 5, wherein the physical characteristic comprises one of mass density or electron density.

7. The computer-implemented method of claim 5, wherein the value for the physical characteristic is mapped to a theoretical attenuation value associated with the first imaging condition.

8. The computer-implemented method of claim 7, wherein the first calibration curve indicates the theoretical attenuation value based on a measured attenuation value for the voxel in the plurality of voxels.

9. A computer-implemented method of detecting an imaging system error in an X-ray imaging system, the method comprising:
using a first imaging condition for the x-ray imaging system, generating a set of projection images of a phantom that includes one or more material inserts;
based on the set of projection images of the phantom, reconstructing a digital volume that includes the one or more material inserts;
for each of the one or more material inserts:
determining a measured attenuation value based on the digital volume; and
determining a corresponding theoretical attenuation value based on properties of the x-ray imaging system and on the first imaging condition for the x-ray imaging system;
based on one or more of the measured attenuation values and one or more of the corresponding theoretical attenuation values, generating a first calibration curve for the X-ray imaging system associated with the first imaging condition;
determining a difference between one or more characteristics of the first calibration curve and one or more characteristics of a second calibration curve that indicates a theoretically expected relationship between the one or more measured attenuation values and the one or more corresponding theoretical attenuation values when the one or more material inserts are imaged by the X-ray imaging system using the first imaging condition; and
in response to the difference exceeding a threshold value, causing a warning indicator to be generated.

10. The computer-implemented method of claim 9, wherein the first imaging condition comprises one or more of a first size of the region of patient anatomy a first imaging energy, or a first X-ray filtration spectrum associated with the X-ray imaging system.

11. The computer-implemented method of claim 9, wherein determining the measured attenuation value for a first material insert of the one or more material inserts comprises:
selecting a region of the digital volume that corresponds to a non-edge portion of the first material insert; and
determining the measured attenuation value for the first material insert based on measured attenuation values associated with the non-edge portion of the first material insert.

12. The computer-implemented method of claim 11 wherein the measured attenuation value for the first material insert comprises an average attenuation value of the measured attenuation values associated with the non-edge portion of the first material insert.

13. The computer-implemented method of claim 9, wherein the first calibration curve for the X-ray imaging system associated with the first imaging condition comprises a first linear curve with a first slope value and a first intercept value.

14. The computer-implemented method of claim 13, wherein the first calibration curve for the X-ray imaging system associated with the first imaging condition further comprises a second linear curve with a second slope value and a second intercept value.

15. The computer-implemented method of claim 13, wherein the difference is based on a first comparison of the first slope value with a second slope value of the second calibration curve and a second comparison of the first intercept value and a second intercept value of the second calibration curve.
